(19) United States
(12) Patent Application Publication
(10) Pub. No.: US 2017/0209084 A1
(43) Pub. Date: Jul. 27, 2017

(54) DIAGNOSIS OF AFFECTIVE DISORDERS USING MAGNETIC RESONANCE SPECTROSCOPY NEUROIMAGING

(71) Applicant: UNIVERSITY OF UTAH RESEARCH FOUNDATION, Salt Lake City, UT (US)

(72) Inventors: Perry F. RENSHAW, Salt Lake City, UT (US); Xian-Feng SHI, Salt Lake City, UT (US); Douglas G. KONDO, Salt Lake City, UT (US)

(21) Appl. No.: 15/308,808

(22) PCT Filed: May 7, 2015
(86) PCT No.: PCT/US15/29651

§ 371 Date: Nov. 3, 2016

Related U.S. Application Data
(60) Provisional application No. 61/990,057, filed on May 7, 2014.

(51) Int. Cl. A61B 5/16; A61B 5/055; A61B 5/00

(52) U.S. Cl. CPC A61B 5/165; A61B 5/0042; A61B 5/055; A61B 5/4076; G01R 33/485

(57) ABSTRACT

A method for facilitating diagnosis of an affective disorder in a person can include (a) acquiring ¹H spectroscopic data to obtain an indicator of total choline (tCho) and creatine (Cr) in an area, e.g., the anterior cingulate cortex, of the person's brain; and (b) determining, by a processor and based on the indicator, at least one of (i) a likely diagnosis of bipolar disorder or major depressive disorder, or (ii) a probability of bipolar disorder or major depressive disorder, in the person.

### CROSS REFERENCE TO RELATED APPLICATIONS

This application claims the benefit of U.S. Provisional Patent Application Ser. No. 61/990,057, filed May 7, 2014, titled DIAGNOSIS OF AFFECTIVE DISORDERS USING MAGNETIC RESONANCE SPECTROSCOPY NEUROIMAGING, which is hereby incorporated by reference in its entirety for all of its teachings.

### STATEMENT REGARDING FEDERALLY FUNDED RESEARCH

This invention was made with government support under 5R21MH096858 awarded by National Institute of Mental Health. The Government has certain rights to this invention.

### BACKGROUND

The subject technology relates to neuroimaging tests to facilitate psychiatric diagnoses, and aid in the differential diagnosis of Major Depressive Disorder (“MDD”) and/or Bipolar Disorder (“BD”) in patients presenting with depressive symptoms.

### SUMMARY

In one embodiment, the invention provides a method for facilitating diagnosis of an affective disorder in a person, including: acquiring ¹H spectroscopic data from a region of interest in the brain of the person; analyzing the data by a processor, to obtain an indicator of total choline (tCho) and creatine (Cr) in a region of the brain; and determining, by a processor and based on the indicator, at least one of (i) a likely diagnosis of bipolar disorder or major depressive disorder, or (ii) a probability of bipolar disorder or major depressive disorder, in the person.

In another embodiment, the invention provides a method of treating a person, including: treating a person, with at least one medication, based on at least one of (i) a likely diagnosis of bipolar disorder or major depressive disorder, or (ii) a probability of bipolar disorder or major depressive disorder, in the person; acquiring ¹H spectroscopic data from a region of interest in a brain of the person; by a processor, analyzing the data to obtain an indicator of total choline (tCho) and creatine (Cr) in a region of the brain; determining, based on the indicators, at least one of the likely diagnosis or the probability; and outputting, by a processor, an indicator of the at least one of the likely diagnosis or the probability.

In a further embodiment, the invention provides a computer-implemented system for identifying bipolar disorder in a person, the system including: a processing module that processes ¹H spectroscopic data, from a region of interest in a brain of the person, so as to obtain an indicator of total choline (tCho) and creatine (Cr) in a region of the brain; and an output module, in communication with the processing module, that outputs, based on the indicators, a machine-readable indicator of at least one of at least one of (i) a likely diagnosis of bipolar disorder or major depressive disorder, or (ii) a probability of bipolar disorder or major depressive disorder, in the person.

Other aspects of the invention will become apparent by consideration of the detailed description and accompanying drawings.

### BRIEF DESCRIPTION OF THE DRAWINGS

The accompanying drawings, which are included to provide further understanding of the subject technology and are incorporated in and constitute a part of this description, illustrate aspects of the subject technology and, together with the specification, serve to explain principles of the subject technology.

FIG. 1 shows a representative proton (¹H) magnetic resonance spectrum of the anterior cingulate cortex at 3 Tesla (TR=2000 ms, TE=135). Cho=Choline; Cr=Creatine; Glx=Glutamine+Glutamate; NAA=N-Acetyl Aspartate; ppm=parts per million. The peak that is labeled “Cho” corresponds to total choline (“t(Cho)”) as described herein.

FIG. 2 is a phosphorus (³¹P) magnetic resonance spectrum of the whole brain at 3 Tesla (TR=3000 ms, TE=2.3 ms). PME=phosphomonoester; Pi=inorganic phosphate; PDE=phosphodiester; PCr=phosphocreatine; NTP=Nucleoside Triphosphate; ppm=parts per million.

FIG. 3 shows mean volume comparisons of cerebrospinal fluid (CSF), gray matter (GM) and white matter (WM) between patients with BD and patients with MDD.

FIGS. 4A-4C show the location of a volume of interest in the ACC region of a human brain for an exemplary method as described herein.

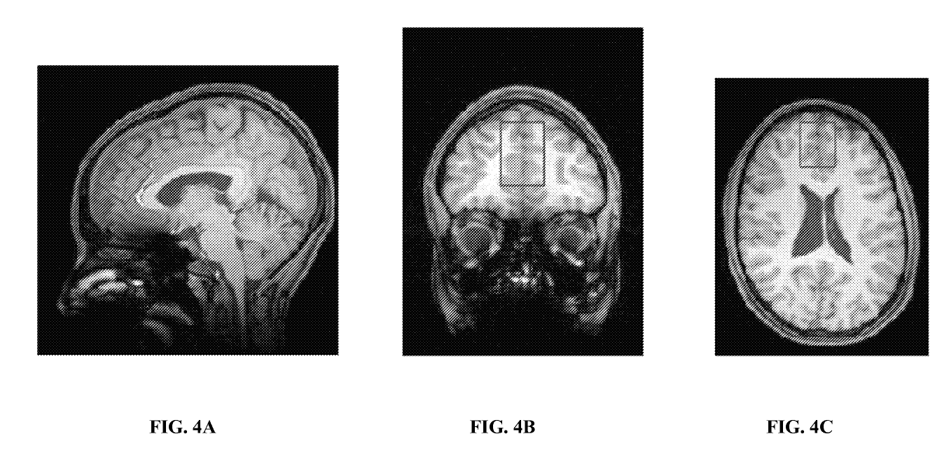

FIGS. 5A-5D show data obtained with an exemplary method as described herein. FIG. 5A shows brain anterior cingulate cortex tCho/Cr (total choline normalized by creatine) levels in bipolar depression (BD) vs. major depressive disorder (MDD) [p=0.0002], as a standard box and whisker plot. The top and bottom of the box identify the first and third quartile, and the solid line within the box represents the median.

FIG. 5B shows a receiver operating characteristic curve, representing how accurately tCho/Cr predicts BD vs. MDD diagnosis. The area under the ROC curve=0.88.

FIG. 5C shows tCho/Cr level versus CDRS score in MDD adolescents (p=0.15) and BD adolescents (p=0.57), respectively.

FIG. 5D shows that adolescents with MDD demonstrated significantly decreased glutamate plus glutamine (“Glx”) level with increasing CDRS score (p=0.02), while in BD adolescents, no significant correlation of Glx level and CDRS score is shown (p=0.63).

FIGS. 6A-6B show illustrations of exemplary methods as described herein. FIG. 6A shows an exemplary design and flowchart for a method according to at least some embodiments of the subject technology.

FIG. 6B shows an additional exemplary design of a method according to at least some embodiments of the subject technology.

FIG. 7 is a simplified diagram of a network of computer systems such as those shown in FIG. 8 for implementing embodiments of the invention.

FIG. 8 is a diagram of a computer system for implementing embodiments of the invention.

### DETAILED DESCRIPTION

It is to be understood that both the foregoing general description and the following detailed description are exemplary and explanatory and are intended to provide further explanation of the subject technology as claimed. The invention is not limited in its application to the details of construction and the arrangement of components set forth in the following description or illustrated in the drawings. The invention is capable of other embodiments and of being practiced or of being carried out in various ways. Additional features and advantages of the subject technology will be set forth in the description below, and in part will be apparent from the description, or may be learned by practice of the subject technology.

In the following detailed description, specific details are set forth to provide an understanding of the subject technology. It will be apparent, however, to one ordinarily skilled in the art that the subject technology may be practiced without some of these specific details. In other instances, well-known structures and techniques have not been shown in detail so as not to obscure the subject technology.

Mood-related problems are common among American adolescents: according to the Centers for Disease Control and Prevention (CDC), 28.5% of U.S. high school students report feeling so sad or hopeless for 2 or more weeks within the past year, that they stopped doing some usual activities, and 15.8% report having seriously considered suicide. Primary care providers and mental health providers can utilize the methods described herein to help guide accurate diagnosis and appropriate treatment for patients presenting with depression symptoms.

Two specific mood disorders, also called affective disorders, are bipolar disorder and major depressive disorder. Bipolar disorder (BD) is a disabling illness and common in persons aged 10-24 years, which represents about 27% of the global population, according to the World Health Organization. BD affects up to 4.5% of adults in the US, and the minimum annual economic burden of BD in the U.S. is about $151 billion. The annual per-patient direct medical costs for BD are greater than that of asthma, diabetes or heart disease.

According to a survey conducted in 2001 by The National Depressive and Manic-Depressive Association Constituency, 69% of BD patients in the U.S. are initially misdiagnosed with major depressive disorder (“MDD”). On average, it is the fourth physician consulted who makes the correct diagnosis of BD. For 35% of Americans with BD, there is a lapse of over 10 years between seeking help and receiving the correct diagnosis. In fact, the median duration of untreated BD is 13 years for patients whose initial mood episode is depressive. Up to 21.6% of primary care patients diagnosed with MDD actually have undiagnosed BD, and delayed diagnosis and/or “unrecognized” cases of BD result in significantly increased costs to the healthcare system.

MDD is a significant global health problem as well, also ranking high on the World Health Organization's list of the most common causes of disability worldwide. MDD, also known as clinical depression or unipolar disorder (UD), and BD together affect up to 20.7% of Americans. Depressive episodes occurring in the course of MDD and BD are two major causes of morbidity and mortality. Similar presenting symptoms make it difficult to distinguish MDD from BD, particularly for non-mental health professionals. The two illnesses are common, require different treatments, are potentially lethal, and are traditionally diagnosed by the current diagnostic standard of care, namely clinical interview. Clinical interviews are incontrovertibly inefficient and require specialized training, expertise, and prolonged interactions with the patient. These approaches are typically not available to primary care clinicians, who write the majority of prescriptions for psychotropic medications.

In the U.S., the National Comorbidity Survey Replication study found that the peak age of onset for mental disorders is 14 years, with MDD most commonly emerging during adolescence. With an annual incidence of 2% in children and 4% to 8% in adolescents, and a cumulative lifetime prevalence of up to 20%, MDD in children is associated with academic failure, social impairment, substance abuse, and suicide attempts. Compared with healthy controls, depressed adults are more likely to have had a depressive episode in adolescence. Adding to the morbidity and mortality experienced by patients and their families, pediatric MDD imposes a substantial economic burden on society. The personal and financial toll is amplified by the fact that only 50% of patients with MDD are diagnosed before reaching adulthood.

Adolescents have been relatively understudied with respect to the neurobiology of mood disorders. Compared with adult depression, adolescents experience more episodes, increased suicidality and greater likelihood of hospitalization. Adolescents with BD may experience more depressive episodes during puberty. Because BD and MDD require different treatments, delayed diagnosis and treatment in bipolar adolescent patients cause significantly increased social and individual burdens. Delayed diagnosis poses a particular problem in females. Women with BD may also experience more depressive episodes. Determining a neurobiological basis of both types of depression, ideally in a manner which allows for diagnosis and/or treatment in adolescents, is a critical unmet need.

In the field of psychiatry, biomarkers are being increasingly investigated. A biomarker is a characteristic that is objectively measured and evaluated as an indicator of normal biological processes, pathogenic processes, or pharmacological responses to a therapeutic intervention. The readiness of biological markers to serve as features, risk factors, or diagnostic criteria is of significant concern in the development of the DSM-V, and the research agenda for DSM-V emphasizes the need to translate research findings into a new classification for psychiatric disorders. In psychiatry, biomarkers could be used to detect and assess or to predict the development of psychiatric disorders, as well as being used to inform treatment decisions. It has been argued that the DSM-V should be structured to permit incorporation of well-replicated findings from neuroscience, by creating mechanisms to flexibly evaluate genetic markers or neuroimaging results rather than waiting for publication of the DSM-VI. A consensus has emerged that advances in the assessment, treatment, and prevention of brain disorders are likely to originate from studies based on clinical and translational neuroscience.

In the search for translational tools, neuroimaging can aid psychiatry in its quest to join the other specialties in medicine in utilizing tests anchored in biology for delivering care and developing new interventions. Advances in child psychiatry neuroimaging provide a scientific infrastructure for understanding numerous psychiatric disorders. Because it can define the neural structures and pathways that mediate illness and its progression, imaging has the potential for use in clinical decision making and disease monitoring. Owing to the fact that depression is not associated with gross tissue pathology or with unambiguous animal models for spontaneous and recurrent episodes, the availability of research tools to noninvasively assess the brain is critical to elucidating the neurobiology of mood disorders. In addition to the scientific insight they provide, the neuroimaging methods employed in child and adolescent psychiatry are noninvasive and have proven to be safe.

Child and adolescent psychiatry investigators have adopted a number of neuroimaging approaches. Some findings have used Magnetic Resonance Spectroscopy (MRS) in the study of pediatric MDD. MRS is a procedure that allows measurement of relevant neurochemistry. Its application to the study of MDD is therefore of particular interest, because mood disorders are illnesses of “state.”

One distinction between magnetic resonance imaging (MRI) and MRS is the type of information the magnetic resonance signal is used to encode. MRI studies create anatomical images whereas MRS provides quantitative biochemical information about the tissue under study. Rather than high-resolution images, MRS data are presented as graphical spectra, with the area under each peak representing the relative concentration of nuclei detected for a given atomic species, for example, hydrogen or phosphorus. The x-axis of graphed MRS data denotes the frequency shift localizing the metabolite in parts per million. MRS spectra peaks correspond with specific chemical compounds of interest. Thus, MRS non-invasively provides a repeatable measure of chemical concentration data in living tissues, including the human brain.

For the patient, the procedure is identical to a clinical study except for the amount of time spent in the scanner; the duration ranges from less than 10 minutes for acquisition of proton spectra to approximately 30 minutes for a phosphorus scan. MRS scans use no radiation, which allows for repeated measurement at different times in the course of a patient's illness, for example, prior to the start of treatment and at the point that remission is achieved. As there is no radiation used in the scan, it is amenable for routine use with adolescents.

As used herein, the term “unmedicated” refers, in some embodiments, to the absence of pharmacological treatments for bipolar disorder or major depressive disorder, such as mood stabilizers like lithium and valproic acid or other psychotropic medications used to treat bipolar disorder or major depressive disorder. Accurate neuroimaging of biomarkers can be confounded through the use of psychotropic medication which affects the brain regions of interest. In contrast, neuroimaging of unmedicated persons (e.g., bipolar disorder patients or major depressive disorder patients) allow researchers to identify biomarkers of both bipolar disorder or major depressive disorder illness and treatment response, without confounding by current treatment.

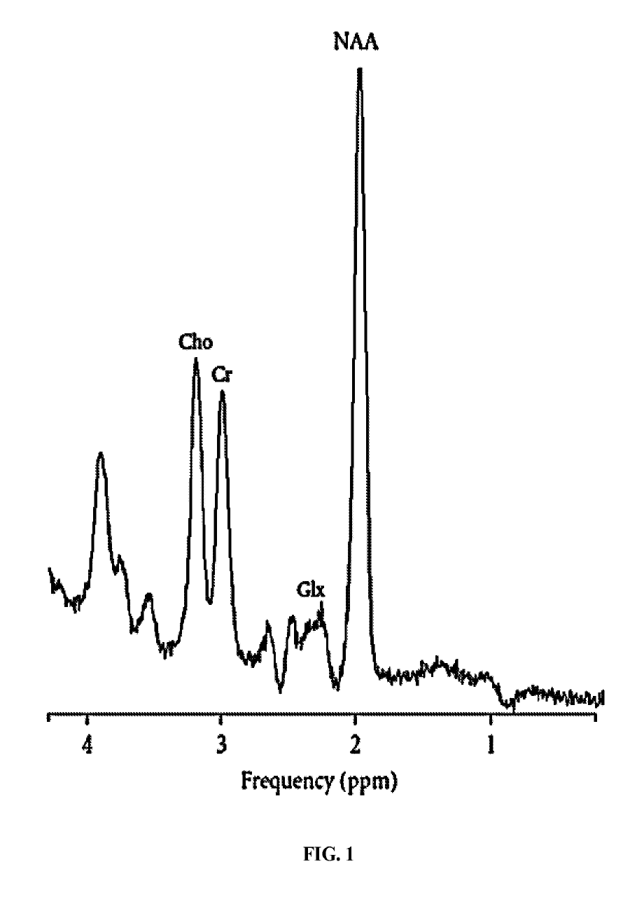

FIG. 1 shows an example of a ¹H-MRS spectrum obtained from a specific area of the anterior cingulate cortex (“ACC”) of a patient with MDD. The chemicals quantifiable with ¹H-MRS include the following: N-acetyl-aspartate (NAA), creatine (Cr), choline (Cho), myoinositol (mI), and lactate (Lac). The location of each peak on the x-axis represents the resonance frequency of the protons in the specific chemical compound, and is referred to as the “chemical shift” of the protons. Each chemical compound that contains protons has a unique chemical shift, which reflects the local environment (i.e. neighboring protons) of the compound. The chemical shift does not vary across properly calibrated MRS instruments, and can be confirmed using known reference samples. The area under the peak is related to the amount of compound present in the area of brain being scanned.

The term “GLX” (Glx) is used to designate the single peak containing the amino acid neurotransmitters glutamate (Glu), gamma-aminobutyric acid (GABA), and glutamine (Gln), because ¹H-MRS signals from Glu and Gln are complicated by the interaction of neighboring protons and the pH dependence of chemical shift. Glu is the major excitatory neurotransmitter in the human brain and was first measured in 1992. Brain in vivo concentrations of Glu are approximately 8-13 times that of GABA, and the ratio of Glu/Gln ranges from 2.4-3.8; therefore, alterations in Glx are typically attributed to altered Glu concentrations. GABA, the major inhibitory neurotransmitter in the brain, has a ¹H-MRS peak that can be separated from Glx at magnetic field strengths ≧2 Tesla using spectral editing technique or 2-D J-resolved spectra.

NAA is the most prominent ¹H-MRS peak and is found only in the nervous system. It is a marker of neuronal density or function, osmoregulation, and energy homeostasis; there is a direct relationship between NAA synthesis, oxygen consumption, and ATP production in the central nervous system. NAA may also play a critical role in myelin production within oligodendrocytes. Reduction in NAA levels measured by ¹H-MRS is a recognized marker of neuronal loss or dysfunction in several psychiatric and neurological disorders including drug abuse, schizophrenia, traumatic brain injury, stroke, epilepsy, multiple sclerosis, neoplasm, HIV encephalopathy, and Alzheimer's disease.

The Cr peak reflects the sum of the creatine and phosphocreatine (PCr) peaks. ¹H-MRS will measure all of the Cr present in the brain, including both the creatine and PCr, while ³¹P-MRS will only measure the amount of PCr. The equilibrium maintained between Cr and PCr is determined by the cellular demand for the high-energy phosphate stored as creatine phosphate. As its level is considered to be relatively constant, Cr may be used as an internal standard for comparison. In this regard, the Cr can be used to “normalize” the Cho signal, to account for any variability in factors such as operator-induced variability and differences due to the equipment used in obtaining the ¹H-MRS data.

The Cho peak contains four membrane- and myelin-related chemicals: phosphorylethanolamine (PE), phosphorylcholine (PC), glycerophosphorylethanolamine (GPE), and glycerophosphorylcholine (GPC), and is also referred to herein as total choline or t(Cho). Cho is a metabolic marker which is indicative of membrane density and integrity, that is, phospholipid synthesis and degradation. Neuropathology characterized by cell membrane breakdown liberates Cho and increases the free Cho pool, contributing to an increased resonance signal in neurodegenerative disorders. In traumatic brain injury, Cho levels increase in relation to the severity of neuronal injury resulting from the breakdown of membranes and myelin. An elevation in the Cho resonance within brain lesions has been accepted as a sign of malignancy. In a ¹H-MRS spectrum, the total Cho or t(Cho) peak has a chemical shift of 3.2 ppm, and arises from the nine protons of the N-trimethyl portion of each choline-containing compound.

Myoinositol (mI) is a sugar involved in the regulation of neuronal osmolarity and the metabolism of membrane bound phospholipids, and is in the phosphoinositide (PI) secondary messenger pathway. Myoinositol is considered a marker of glial proliferation, and an increase in mI resonance may be a proxy for increased inflammation in the brain.

Under normal circumstances, lactate (Lac) is present in the brain at concentrations too small to be detected using ¹H-MRS. However, if the aerobic oxidation mechanism fails and anaerobic glycolysis is triggered—such as brain ischemia, hypoxia, seizure activity, and metabolic disorders—Lac levels rise significantly. Typically, the Lac peak can be observed as an inverted doublet at an echo time of 135 ms at 1.3 ppm. It has been shown that lactate becomes elevated if large numbers of inflammatory cells are activated. In the past two decades, ¹H-MRS has progressed from the laboratory into routine use in the treatment of cancers of the brain and prostate and, in various embodiments, ¹H-MRS may also be used to diagnose mood disorders.

³¹P-MRS is a related neuroimaging method that acquires the resonance spectra of phosphorus rather than hydrogen. Although MRS can be performed on a variety of nuclei such as carbon, nitrogen, fluorine, and sodium, only the nuclei of phosphorus (³¹P) and hydrogen (¹H) exist in vivo in sufficient concentrations for routine clinical evaluation. Studies employing ³¹P-MRS have indicated possible abnormalities in membrane high-energy phosphate metabolism, phospholipid metabolism, and intracellular pH in mood disorders. The methods described by Iosifescu et al, Biol. Psychiatry 2008, vol. 63, pp. 1127-1134, are incorporated herein by reference in their entirety.

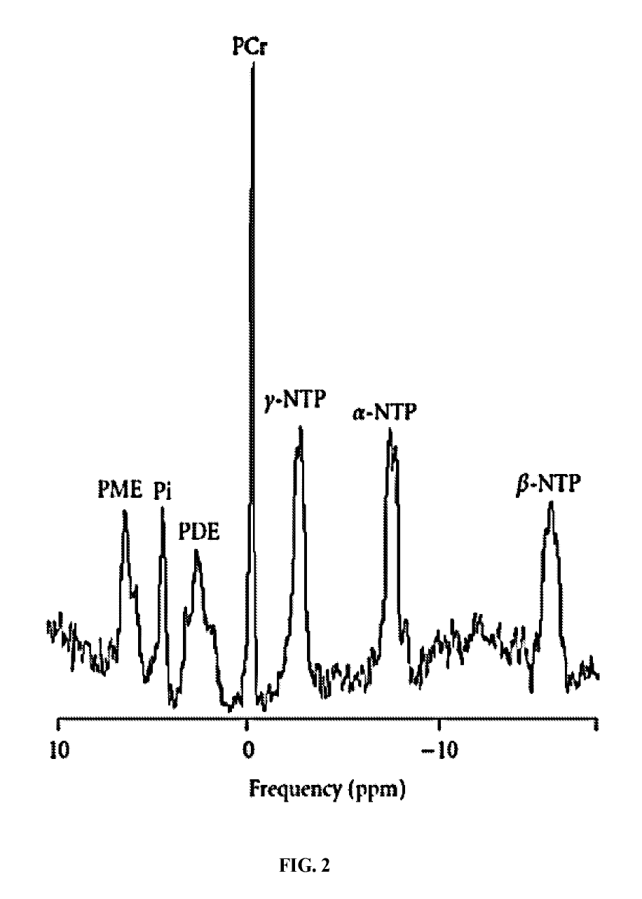

In ³¹P-MRS spectra of the brain, seven chemical peaks are resolved; these are phosphomonoester (PME), inorganic phosphate (Pi), phosphodiester (PDE), phosphocreatine (PCr), and alpha-(α), beta-(β), and gamma-(γ) nucleoside triphosphate (NTP). FIG. 2 displays an example of the spectra that is acquired with ³¹P-MRS. The β-NTP peak is measured as a proxy for ATP, the principal energy source in brain. The phosphomonoester (PME) peak contains the signals from numerous metabolites, including those related to membrane phospholipid synthesis such as phosphocholine (PC) or phosphoethanolamine (PE) and sugar phosphates such as glycerophosphate or inositol phosphates. In the PME region, PE is the most abundant and PC is the second most abundant metabolite.

The membrane breakdown products glycerophosphocholine and glycerophosphoethanolamine contribute to the PDE peak, but most of the signal in the in vivo PDE peak arises from membrane phospholipid itself, making PDE as a marker of neuronal integrity. The Pi peak appears between the PME and PDE peaks. Pi appears in many metabolic pathways. Although the Pi peak contains both PO₄⁻¹ and PO₄⁻², these two forms of Pi register as a single peak due to the rapid exchange between these two molecules, although the position of this single peak may shift depending on the relative abundance of each of the PO₄⁻¹ form relative to the PO₄⁻² form of Pi. Since the position of the single peak reflects the equilibrium between PO₄⁻¹ and PO₄⁻², this allows investigators to calculate brain pH from the chemical shift of the Pi peak. Because the phosphate ions exist in the intracellular space, this calculated pH reflects intracellular pH (pHi).

The PCr peak is the most prominent peak in the ³¹P-MRS spectra in the brain. PCr conveys high-energy phosphates from the mitochondria to the cytosol. When an ATP molecule is consumed, PCr transfers its high-energy phosphate group to ADP (adenosine diphosphate), thus replenishing ATP via the creatine kinase reaction. In this regard, PCr behaves as a buffer of ATP. PCr is abundant in tissues with rapidly varying energy demands, that is, brain and muscle tissue. This ATP buffer is absent in tissues where energy demands are constant, such as liver tissue.

NTP forms three distinct peaks; alpha (α), beta (β), and gamma (γ) nucleoside triphosphate, of which the doublet of the γ-ATP peak is resolved in ³¹P-MR spectra. ATP is the bioenergetic substrate for many biochemical processes in the brain and is present at a much higher concentration, on the order of 1.8 mM, than any other NTP.

Proton (¹H) spectroscopy has been employed MRS method used in psychiatric research. Protons are abundant in organic structures, and their nuclei have high magnetic sensitivity. In 1995, ¹H-MRS became widely available when the U.S. Food and Drug Administration (FDA) approved the software for an automated and inexpensive MRS sequence protocol, the PROton Brain Examination (PROBE), which can be run without dedicated research personnel on a standard MRI scanner. The majority of ¹H-MRS studies have been conducted using MRI scanners operating at magnetic field strengths of 1.5 Tesla or less, which is less than optimal for ³¹P-MRS. However, 3 Tesla MRI was approved by the FDA in 2000 and is becoming more accessible to major medical centers. The increased availability of scanners with 3 or 4 Tesla magnetic fields will improve the sensitivity of MRS studies.

At present, to conduct MRS brain scans that target other nuclei of interest to psychiatrists, such as phosphorus or lithium, specialized equipment and research expertise is required (although not a separate MRI machine). These obstacles will not be insurmountable if ³¹P-MRS, in particular, proves to be a valid and reliable measure of one or more translational biomarkers in the affective disorders. To date, unlike physicians in other fields of medicine, psychiatrists do not benefit from working with objective measures of illness and recovery, such as blood pressure or hemoglobin A1c. However, identification of a validated biomarker for use in clinical psychiatry would be beneficial, and MRS investigators have joined the pursuit along with their colleagues in genetics, neuroscience, and other branches of neuroimaging.

In recent years, multiple studies have reported regional and global hypometabolism in subjects experiencing a major depressive episode, which could be related to the pathophysiology of mood disorders. Abnormalities of bioenergetic metabolism in adults have been described, primarily decreased baseline levels of β-nucleoside triphosphate and total NTP, in the basal ganglia and the frontal lobes of MDD subjects compared with healthy control subjects. MRS studies can provide investigators with a robust methodology to test specific hypotheses regarding the neurobiology of mood disorders in adolescents as well as adults.

In vivo MRS is a noninvasive imaging technique which is capable of directly assessing the living biochemistry in localized brain regions. Studies of depressed children and adolescents have a number of advantages compared with studies of adults: the effects of statistical covariates such as repeated episodes, duration of illness, multiple medications, and normal aging are avoided. Thus, MRS may be a translational research tool capable of partially obviating the developmental and environmental confounders that have made research in child psychiatry a difficult challenge.

MRS findings implicate Glx, NAA, and choline (including its correlation with immune system metabolites) in the neurobiology of MDD. Altered levels of choline would be consistent with altered neural plasticity as well as animal models of depression and antidepressant response.

According to some embodiments, repeated MRS scans can be used to ascertain whether there are changes in neurometabolite concentrations when a patient with MDD responds to treatment, that is, whether the baseline differences in Glx, NAA, and choline are differences of “state” or “trait.” In an embodiment, the patient is a child or an adolescent. Results from such scans can inform researchers regarding the mechanisms by which antidepressants or other psychotic medications work and may provide new treatment targets for drug development. In addition to pointing toward the mechanisms of illness recovery (i.e., the “mediators”), neuroimaging studies can identify predictors of treatment outcome (i.e., the “moderators”). Identification of the mediators and moderators of pediatric MDD treatment would aid in moving toward personalized care.

Contemporary understanding of the neurobiology of depression is focused on imbalances in neural circuits, cellular plasticity and resilience, and impaired neurotrophic signaling cascades. As a research tool, MRS can perform in vivo quantification of the neurometabolite indicators of neuronal integrity, mitochondrial functioning, cellular membrane turnover, and signaling cascades. MRS is an excellent method for in vivo measurement of GABA concentrations, where GABA is understood to play an increasingly central role in our conceptualization of mood disorders and can play a major role in delineating the neurobiology of MDD.

As demonstrated herein, BD can be distinguished from MDD by MRS. In various embodiments, patients with both BD and MDD will be followed and scanned longitudinally (i.e. over a period of time), MRS will be able to test the hypothesis that the major depressive episodes experienced by both groups of patients have the same neurochemical basis. In certain embodiments, the patients will be children or adolescents.

The ability of MRS to measure neurochemical changes that parallel changes in patients' clinical presentation is established in children and adults. Additional study of children and adolescents will help determine whether differences in depressed patients are the result of altered development across the life span, or if they can be documented early in development, serving as a risk factor for which prevention strategies can be employed.

The subject technology relates to neuroimaging tests, using Magnetic Resonance Spectroscopy (MRS), that are designed to aid in the differential diagnosis of MDD and/or BD in patients presenting with depressive symptoms. The anterior cingulate cortex (ACC) is thought to be important in mediating the symptoms of affective disorders processing. The ¹H-MRS metabolite concentration from the brain ACC region among BD and MDD female adolescents can be evaluated as disclosed herein. In certain embodiments, the evaluated brain region is selected from at least one of the ACC, the dorsolateral prefrontal cortex, and the amygdala. In an embodiment, the evaluated brain region is the prefrontal cortex. In a further embodiment, the evaluated brain region is the ACC.

According to some embodiments, MRS can be used to help clinicians assess the effects of medication. In certain embodiments, MRS can be used to help clinicians diagnose an affective disorder. In further embodiments, MRS can be used to help clinicians assess the probability that a patient has an affective disorder. The results of the MRS analysis can be used to guide treatment or therapy appropriate to the patient. In some embodiments, MRS analyses such as those disclosed herein may be used to select patients for studies such as clinical trials by identifying patients with particular conditions (e.g. BD or MDD) that are targets for a drug and/or therapy in the study or trial, or by identifying patients that are more likely to respond to treatments (e.g. by selecting patients who exhibit larger changes in indicator levels upon treatment, e.g. medication).

The specificity of MRS in mood disorders suggests that MRS can identify neurochemical differences between mood disorders and OCD, Intermittent Explosive Disorder, and Attention-deficit Hyperactivity Disorder (ADHD).

An illustrative example of the potential for specificity offered by MRS is the case of Glx and ADHD. Investigators have found elevated Glx in brain regions of interest in ADHD patients compared to healthy controls. Some found increased Glx in the right prefrontal cortex and striatum of ADHD subjects, others documented increased Glx in the left and right frontal lobes, and yet others found elevated Glx concentrations in the striatum treatment-naïve ADHD patients. In addition, others have shown that changes in glutaminergic tone occur with ADHD treatment. In contrast to the findings in ADHD, Glx has been shown to be reduced in MDD, and pediatric BD with and without ADHD is differentiated by Glx concentrations in the anterior cingulate cortex.

According to some embodiments, techniques of the subject technology may be combined with other diagnostic tools to provide a diagnosis. For example, a patient may be evaluated initially and/or after treatment based on a standardized diagnostic interview.

For example, the psychiatric differential diagnosis tools used at baseline, when patients first enter the studies can be, for patients 13-17 years old, a standardized diagnostic interview called the K-SADS-PL (Kiddie Schedule for Affective Disorders and Schizophrenia-Present and Lifetime Version), developed at the University of Pittsburgh; or, for patients 18-20 years old, a standardized diagnostic interview called the SCID (Structured Clinical Interview for DSM Disorders). Both the K-SADS-PL and SCID are widely-accepted as valid, and are commonly used instruments in published studies.

Under most circumstances, one would expect symptoms to change, rather than the patients' diagnosis during a study or during treatment. So to measure the effect of interventions vs. placebo in reducing the severity of depressive symptoms over, for example, 6-8 weeks, depression rating scales that have been psychometrically validated can be administered. Such depression rating scales have been shown to be sensitive to change in psychiatric treatment studies. For patients 13-17 years old, the CDRS-R (Children's Depression Rating Scale-Revised) can be used. For patients 18-20 years old, the MADRS (Montgomery-Asberg Depression Rating Scale) can be used.

The MRS data obtained from a patient may provide an indicator of total choline (tCho) and creatine (Cr) levels in the evaluated brain region. In an embodiment, the indicator is the ratio of tCho divided by, or to, Cr. In some embodiments, the indicator is the ratio of tCho to PCr. In certain embodiments, the indicator is correlated with at least one of tCho, Cr, Glx, NAA, NAAG, mI, Lac, PCr, PME, Pi, PDE, or NTP. In some embodiments, the indicator is correlated by at least two of tCho, Cr, Glx, NAA, NAAG, mI, Lac, PCr, PME, Pi, PDE, or NTP. In further embodiments, the indicator is correlated by at least three of tCho, Cr, Glx, NAA, NAAG, mI, Lac, PCr, PME, Pi, PDE, or NTP. Alternatively, the indicator is correlated by data obtained by ³¹P-MRS. Further, the indicator is correlated by data obtained by ¹H-MRS.

According to some embodiments, evaluation of an indicator of total choline (tCho) and creatine (Cr) levels, such as tCho/Cr levels, occurs via an initial scan that would be performed when a patient's mood is depressed but their diagnosis is unclear. In certain embodiments, the type of data obtained by MRS for use in evaluating a patient is at least one of tCho, Cr, Glx, NAA, NAAG, mI, Lac, PCr, PME, Pi, PDE, NTP, or any combination thereof. In some embodiments, the type of data used for evaluating a patient is obtained by ¹H-MRS. In alternative embodiments, it is obtained by ³¹P-MRS. Evaluation of a patient may occur when the patient does not have, or exhibit any symptoms of, an affective disorder.

The results of the scan can be useful to the diagnosing physician so as to provide the correct treatment, or to inform the physician about the likelihood or probability that the patient may have an affective disorder. Once the patient has received a treatment, the mood state may improve, for example, from depressed to euthymic.

A second scan may be performed after treatment is applied. If an indicator of total choline (tCho) and creatine (Cr) levels, such as tCho/Cr, on a second, post-treatment scan was “improved” in the major depression patients, the tCho/Cr measurement may be no different from that of a healthy control patient and/or a patient with an affective disorder, such as BD.

Alternatively, the determination of whether a prescribed course of treatment is effective can be assessed without a second scan.

Because MDD and BD require different treatments, discovery of methods for accurately distinguishing MDD and BD represents a critical unmet need. During adolescence, the rate of depression in females rises to approximately twice that found in males (prevalence ratio=1.3-3.1; lifetime gender ratio=2.1). This finding is robust to international sampling across continents and cultures, and has been repeatedly replicated. Thus, diagnostic methods for females during the critical adolescent period of development are urgently needed. Proton magnetic resonance spectroscopy (¹H-MRS) is a safe, noninvasive neuroimaging method for performing quantitative measures of individual patients' brain chemistry.

In certain embodiments, ¹H-MRS brain scans can be performed on study participants at baseline, and then repeated following several weeks of treatment. This allows for a comparison of the brain chemistry of MDD with that of BD. As described herein, ¹H-MRS was used to acquire data from a voxel located in the anterior cingulate cortex (ACC), using a standard PRESS pulse sequence. A comparison was made between tCho (normalized by creatine) in depressed female adolescents with BD (n=9; mean age=17.3 years) and MDD (n=28; mean age=17.0 years). Statistical analysis revealed significantly elevated tCho levels in MDD compared to BD (p=0.0002).

The volume of the brain region evaluated by MRS may vary. In certain embodiments, the voxel of interest is about 18.75 cm³. In further embodiments, the voxel is about 3 cm³, about 8 cm³, or about 20 cm³. In some embodiments, the voxel of interest is between about 1 cm³ and about 50 cm³, between about 5 cm³ and about 40 cm³, between about 15 cm³ and about 20 cm³, at least about 10 cm³, or not more than about 30 cm³. In certain embodiments, the volume of the brain region evaluated by MRS may include one or more voxels. MRS techniques used in various embodiments include: proper and reproducible subject positioning, frequency and field homogeneity shimming, acquisition of pilot images, slice positioning and shimming, imaging of necessary adjacent slices, and selection of appropriate pulse sequences, acquisition parameters, and data analysis processing algorithms. Actual scan time for a standard ¹H-MRS spectrum is on the order of 7 to 10 minutes, although shorter or longer scan times may be used.

In addition, the ¹H MRS neuroimaging data were subjected to Receiver Operating Characteristic (ROC) analysis. The area under the ROC curve is an expression of how accurately tCho was able to predict participants' MDD vs. BD diagnosis. Utilizing ROC methodology, tCho had a diagnostic accuracy of 88.3% (85.7% sensitive, 77.8% specific).

The differential diagnosis of adolescent MDD and BD is challenging, resulting in significant, multiyear delays to receiving the correct diagnosis and treatment for patients with BD. Elevated tCho levels in MDD vs. BD, ascertained using ¹H MRS, could serve as a diagnostic biomarker in patients presenting with depressive symptoms. By facilitating rapid establishment of an accurate diagnosis, the test would speed delivery of appropriate treatment for these two common and potentially lethal brain diseases.

### EXAMPLES

### Example 1

Nineteen (19) subjects with BD (N=9; mean age 17.3±3.1 years) and 28 subjects with MDD (N=28; mean age 17.0±2.1 years) were recruited, each being a female adolescent.

Data acquisition and processing. All studies were performed on a 3 T clinical MRI system (Trio-Tim, Siemens Medical Solutions, Erlangen, Germany) with Avanto gradients (40 mT/m strength and 150 T/m/s slew rate) using a 12-channel head coil. ¹H spectra were acquired using a PRESS pulse sequence with voxel of 18.75 cm³, TR/TE 2000/31 ms, receiver bandwidth 1 kHz, averages of 64 and vector size 1024. The signal intensity of each metabolite was obtained using LCMODEL fitting.

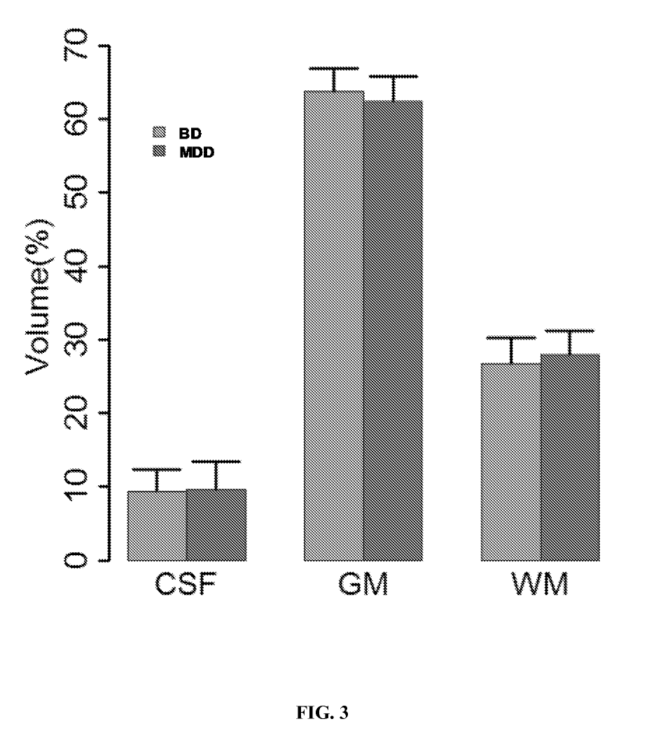

FIG. 3 displays the volume percentage difference of gray matter, white matter, and cerebrospinal fluid between BD and MDD patients in the volume of interest.

Data were acquired from an anterior cingulate cortex (ACC) voxel in the midsagittal plane using a standard PRESS pulse sequence. All measured ¹H-MRS metabolite levels and clinical information are listed in Table 1, which shows ¹H-MRS anterior cingulate cortex metabolite levels in bipolar disorder groups and major depressive disorder groups. The MDD patients are labeled as “UD” in Table 1.

TABLE 1
Metabolite levels and clinical information for depressed subjects.

| | | | P value |
|---|---|---|---|
| | BD (n = 9) | UD (n = 28) | (ANOVA) |
| Mean age (yrs) | 17.3 ± 3.1 | 17.8 ± 2.1 | 0.71 |
| CDRS Score | 55.4 ± 9.6 | 57.3 ± 6.6 | 0.52 |
| MADRS Score | 25.8 ± 9.0 | 25.6 ± 5.8 | 0.96 |
| GM (%) | 63.84 ± 3.04 | 62.46 ± 3.36 | 0.28 |
| WM (%) | 26.80 ± 3.38 | 27.95 ± 3.19 | 0.36 |
| CSF (%) | 9.36 ± 2.96 | 9.59 ± 3.80 | 0.87 |
| tCho/Cr | 0.22 ± 0.02 | 0.25 ± 0.02 | 0.0002 |
| Glx/Cr | 1.56 ± 0.09 | 1.66 ± 0.21 | 0.18 |
| (NAA + NAAG)/Cr | 1.27 ± 0.14 | 1.32 ± 0.13 | 0.36 |
| Cr | 6.11 ± 0.43 | 6.10 ± 0.45 | 0.96 |

No significant difference was observed in age, CDRS score, MADRS score, or GM/WM/CSF volume between BD adolescents and MDD adolescents.

Table 2 shows comparative data for 27 healthy control subjects, with 10 male and 17 female subjects, shown below, measured in the same manner as the subjects with affective disorders. The mean age (yrs) of the healthy male subjects was 17.3±2.4, and for the healthy female subjects was 19.6±2.3.

TABLE 2
Metabolite levels and clinical information for healthy
control subjects.

| | Male (n = 10) | Female (n = 17) |
|---|---|---|
| tCho/Cr | 0.241 ± 0.023 | 0.235 ± 0.018 |
| NAA + NAAG/Cr | 1.329 ± 0.095 | 1.337 ± 0.060 |
| Glx/Cr | 1.554 ± 0.113 | 1.503 ± 0.122 |
| Glx/tCho | 6.481 ± 0.580 | 6.427 ± 0.633 |
| Cr | 6.578 ± 0.399 | 6.703 ± 0.451 |
| tCho | 1.584 ± 0.164 | 1.571 ± 0.122 |
| Glx | 10.187 ± 0.412 | 10.043 ± 0.664 |
| NAA + NAAG | 8.711 ± 0.356 | 8.948 ± 0.548 |

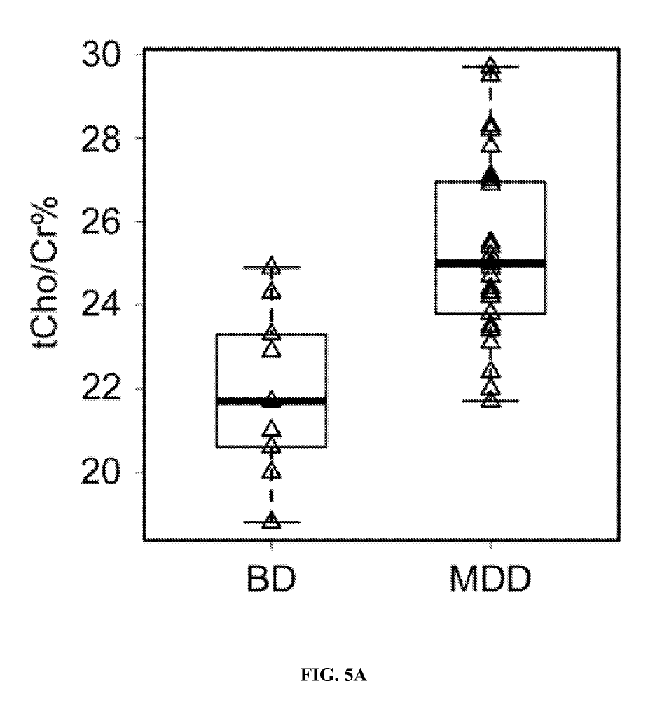

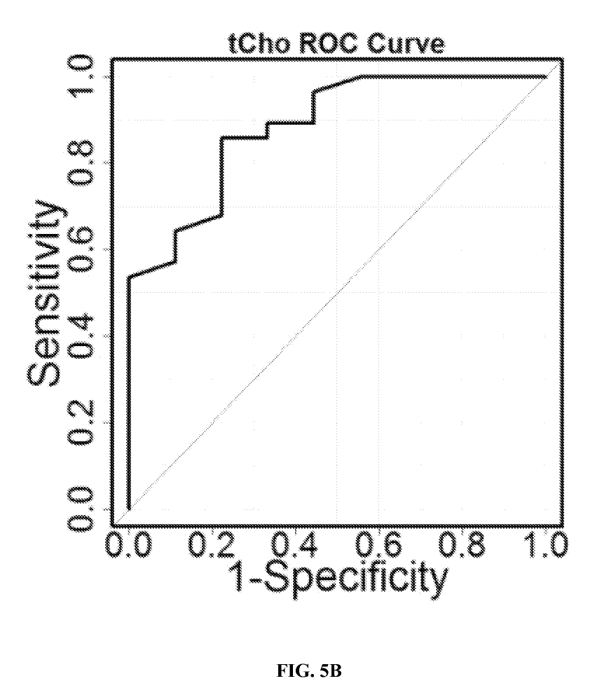

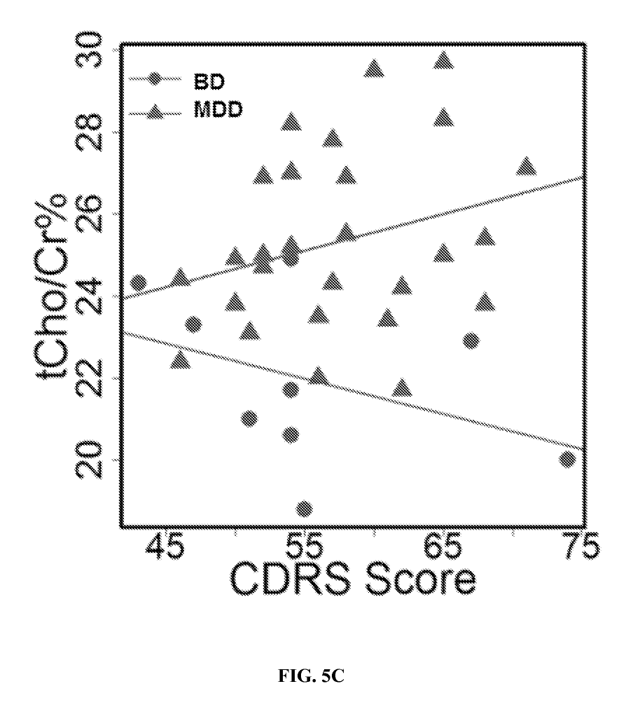

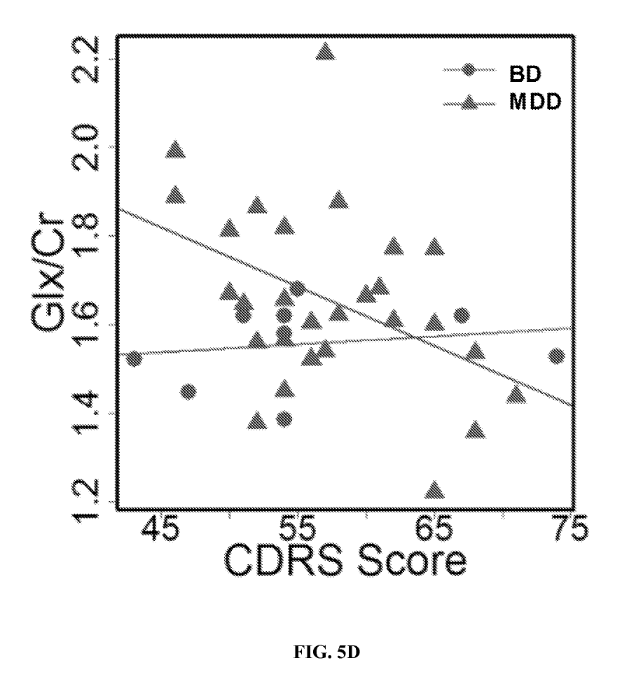

FIG. 5A demonstrates the comparison in tCho (normalized by creatine), between female adolescents with BD and female adolescents with MDD. Statistical analysis revealed a significantly elevated tCho level in the ACC of female adolescents with MDD compared to those with BD (p=0.0002).

The area under the receiver operating characteristic (ROC) curve, shown in FIG. 5B, is an expression of how efficiently the ACC tCho level differentiates the BD and MDD subjects. tCho levels predicted BP and MDD adolescent subjects' diagnosis with an accuracy of 88.3% (85.7% sensitive; 77.8% specific). In addition, correlational analysis of tCho vs. CDRS scores was performed in the BD and MDD groups. Although both group regressions do not reach significance relative to CDRS scores, the respective slopes of BD˜CDRS and MDD˜CDRS demonstrate a trend toward statistical significance (p=0.08) as shown in FIG. 5C. However, significant correlation between the ratio of Glx over Cr and the CDRS score was noted in MDD adolescents instead of BD adolescents (FIG. 5D).

The area under the receiver operating characteristic (ROC) curve of FIG. 5B provides efficacy comparable to other diagnostic tests, as shown in Table 3 below:

The differential diagnosis of MDD and BD in adolescents is challenging, causing significant delays to appropriate diagnosis and treatment for BD patients. The present finding of elevated indicators of total choline (tCho) and creatine (Cr) levels in patients with MDD can serve as a diagnostic biomarker in pediatric major mood disorders.

The data from Tables 1 and 2 indicate that there are no significant differences in the metabolite information between female and male subjects. It is expected that there will be no significant differences between subjects of various ages, as well, and that the trends shown in this Example are independent of age and gender.

The subject technology described herein can be used for systems and methods to facilitate both the diagnosis and treatment of a person with an affective disorder. For example, data can be obtained by a MRS, including both ¹H and ³¹P data, on healthy subjects, to provide a baseline, control or “normal” data set. Similar data can be collected on subjects who have been previously diagnosed with an affective disorder, including BD and MDD, to provide a BD data set and a MDD data set. The affective disorder data sets may be compared with the data sets obtained with healthy subjects, for example, using ANOVA or a multiple regression statistical analysis. The data sets may be used to form a database of information, containing normal data sets and affective data sets, or combinations thereof

Any of the data collected by MRS may be compared. In an embodiment, an indicator of total choline (tCho) and creatine (Cr) levels is compared. In certain embodiments, the tCho/Cr ratio is compared. In further embodiments, an absolute value of tCho is measured and/or determined, and compared. In an additional embodiment, more than one type of data is compared, for example, tCho and PCr. Alternatively, a single type of data may be compared, or at least two types of data may be compared. The comparison of data obtained by MRS can result in determining a diagnosis of an affective disorder, or a probability of having an affective disorder, using a computer processor to aid in the analysis and determination steps of the method or system.

In some embodiments, the ratio of tCho/Cr for a person diagnosed with, or with greater than a 50% probability of having, BD is between about 0.20 and about 0.24; in other embodiments the ratio is between about 0.21 and about 0.23. In certain embodiments, the ratio is about 0.22. In further embodiments, the ratio is less than about 0.24, less than about 0.23, or less than about 0.225.

In some embodiments, the ratio of tCho/Cr for a person diagnosed with, or with greater than a 50% probability of having, MDD is between about 0.23 and about 0.27; in other embodiments the ratio is between about 0.24 and about 0.26. In certain embodiments, the ratio is about 0.25. In further embodiments, the ratio is greater than about 0.23, greater than about 0.24, or greater than about 0.235.

In certain embodiments in which absolute value of tCho is determined, absolute tCho values for a person having MDD are greater than absolute tCho values for a person having BD.

In some embodiments, MRS data may be collected from a person with an unknown mental state, and compared to the normal and/or affective data sets. In a particular embodiment, the person may be suspected of having an affective disorder, such as being identified by a primary care physician who recommends the MRS analysis, or by a patient reporting feeling depressed. The data for the person with an unknown mental state may be used to diagnose an affective disorder. If this occurs, psychiatric medication may be prescribed for, or given to, the person. For example, if at least one of the MRS data values for the person with an unknown mental state is similar, within a specific range of variability, to at least one of the MRS data values in the BD data set, the person may be diagnosed with BD. In an embodiment, the specific range of variability is within 1%, 3%, 5%, 8%, 10%, 15%, 20%, 25%, 30% or 50% of the values of the affective data set. In certain embodiments, the at least one of the MRS data values may be compared to the values of the normal data set, with a specific range of variability within 1%, 3%, 5%, 8%, 10%, 15%, 20%, 25%, 30% or 50% of those values. In certain embodiments, at least one of the MRS data values for the person with an unknown mental state is similar, within a specific range of variability, to at least one of the MRS data values in the MDD data set, resulting in a diagnosis of MDD. In further embodiments, at least one of the MRS data values in the normal and/or affective data set may exhibit a normal distribution, and the variability between the MRS data value of the person with an unknown mental state may be within a specific confidence level, such as within an 80% confidence level, a 90% confidence level, a 95% confidence level, or a 99% confidence level.

In an embodiment, the data for the person with an unknown mental state may be used to calculate the probability of having, or exhibiting symptoms of, an affective disorder. In an embodiment where the range of probabilities is defined as being from zero (the minimum probability of having an affective disorder) to one (the maximum probability of having an affective disorder), a comparison of the data provides a number between, and inclusive of, 0 and 1. If the number is above 0.5, the person with an unknown mental state is more likely than not to have an affective disorder, or greater than a 50% probability. If the number is below 0.5, the person has a lower probability of having an affective disorder, or less than a 50% probability.

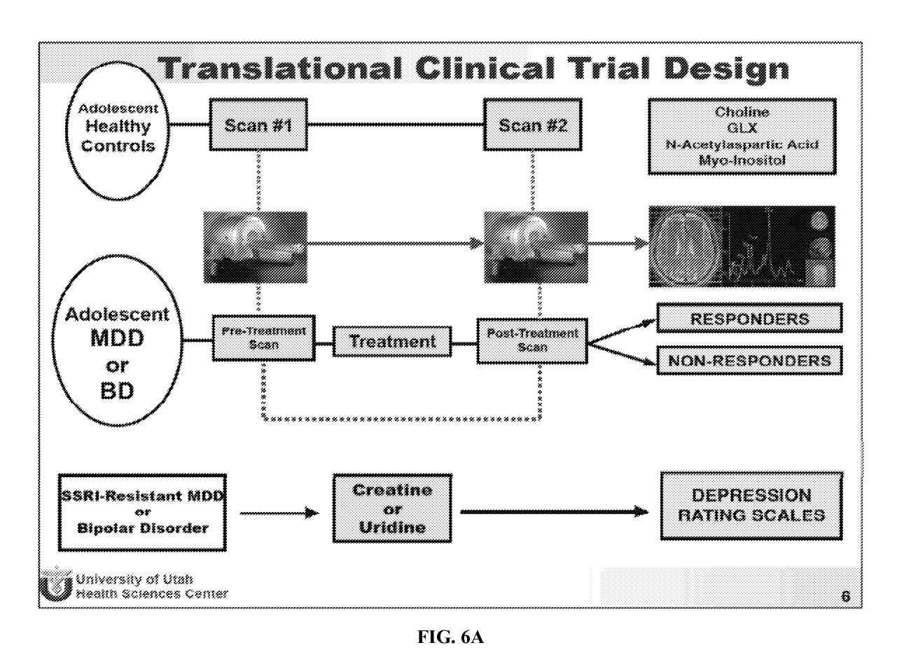

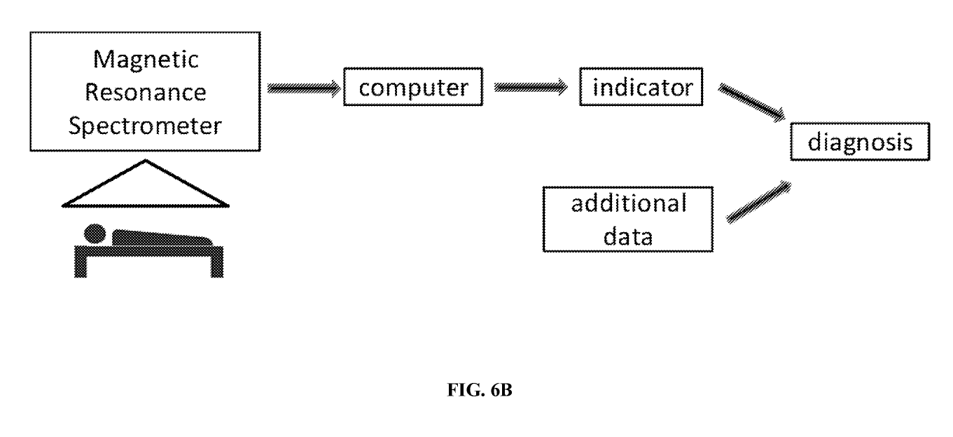

Exemplary systems and methods of diagnosing an affective disorder are illustrated in FIGS. 6A and 6B. FIG. 6A shows a design and flowchart of a method of facilitating diagnosis of, or treating, an affective disorder by obtaining MRS data from a person before and after treatment. In an embodiment, the data may be compared to data from healthy control persons. Treatment may include creatine, T3, or uridine therapy, or a combination thereof. In certain embodiments, treatment includes administering a mood stabilizer, including one selected from at least one of lithium, valproic acid, lamotrigine, or an atypical antipsychotic agent. In some embodiments, treatment includes administering an antidepressant, including an SSRI. Response to the treatment may be monitored by MRS.

FIG. 6B shows an additional embodiment of a method according to the subject technology. A person is scanned by a MRS to obtain data, which may include one or both of ¹H and ³¹P data. A computer processor is used to calculate an indicator of total choline (tCho) and creatine (Cr) levels, which may be expressed as a tCho/Cr ratio. The indicator is compared with additional data, such as a normal dataset, a BD dataset, or a MDD dataset, also using a computer processor. The comparison of the indicator with the additional data can provide an output which provides a diagnosis of the person. In certain embodiments, the output is a probability that the person has an affective disorder. The method may also include a step of treating the person based upon the output. Treatment may include treatment with antidepressants if the person is diagnosed with MDD, or may include treatment with at least one mood stabilizer if the person is diagnosed with BD.

The indicator of tCho and Cr may represent a signal intensity or concentration of each compound, or a ratio of concentrations. The tCho and/or Cr concentration may be an index concentration obtained from a person with an unknown mental state, which may be compared with a threshold concentration obtained from the MDD or BD data set, or from the normal data set.

In certain embodiments, the methods disclosed herein are directed toward facilitating diagnosis of an affective disorder in a person or treating a person, including the steps of acquiring ¹H spectroscopic data and obtaining an indicator of tCho and Cr in a region in the brain by analysis of that data. In further embodiments, the methods include the steps of providing ¹H spectroscopic data and obtaining an indicator of tCho and Cr in a region in the brain by analysis of that data. Thus, the analysis of the ¹H spectroscopic data and obtaining the ¹H spectroscopic data may be performed as distinct steps. In additional embodiments, the methods may not include a step regarding how the ¹H spectroscopic data is obtained.

At least one aspect of the disclosure includes a non-transitory machine readable medium encoded with instructions executable by a processing system to perform one or methods described herein.

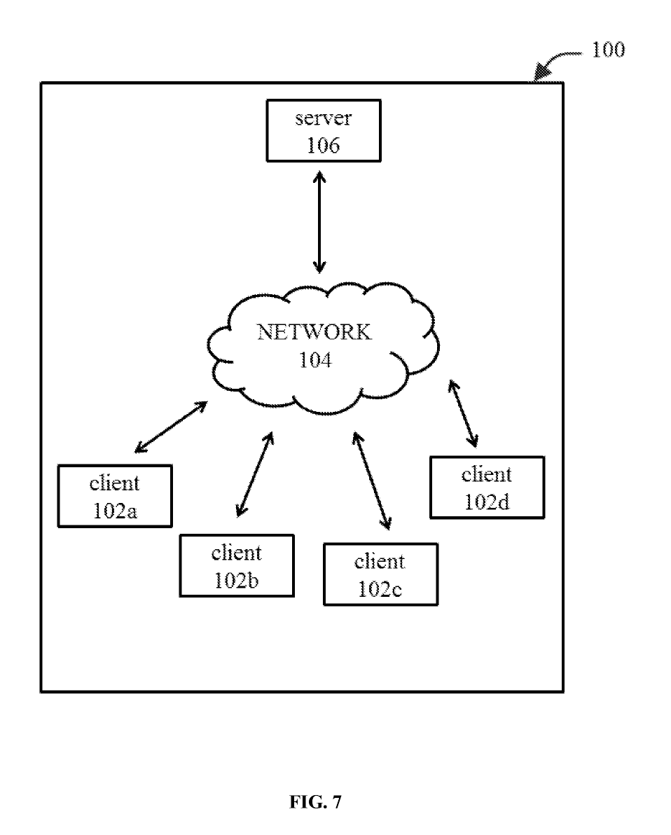

FIG. 7 is a simplified diagram of a system 100, in accordance with various embodiments of the subject technology for carrying out embodiments of the present invention. The system 100 may include one or more remote client devices 102 (e.g., client devices 102a, 102b, 102c, 102d, . . . ) in communication with one or more server computing devices 106 (e.g., one or more servers) via network 104. In some embodiments, a client device 102 is configured to run one or more applications based on communications with a server 106 over a network 104. In some embodiments, a server 106 is configured to run one or more applications based on communications with a client device 102 over the network 104. In some embodiments, a server 106 is configured to run one or more applications that may be accessed and controlled at a client device 102. For example, a user at a client device 102 may use a web browser to access and control an application running on a server 106 over the network 104. In some embodiments, a server 106 is configured to allow remote sessions (e.g., remote desktop sessions) wherein users can access applications and files on a server 106 by logging onto a server 106 from a client device 102. Such a connection may be established using any of several well-known techniques such as the Remote Desktop Protocol (RDP) on a Windows-based server.

By way of illustration and not limitation, in some embodiments, stated from a perspective of a server side (treating a server as a local device and treating a client device as a remote device), a server application is executed (or runs) at a server 106. While a remote client device 102 may receive and display a view of the server application on a display local to the remote client device 102, the remote client device 102 does not execute (or run) the server application at the remote client device 102. Stated in another way from a perspective of the client side (treating a server as remote device and treating a client device as a local device), a remote application is executed (or runs) at a remote server 106.

By way of illustration and not limitation, in some embodiments, a client device 102 can represent a desktop computer, a mobile phone, a laptop computer, a netbook computer, a tablet, a thin client device, a personal digital assistant (PDA), a portable computing device, and/or a suitable device with a processor. In one example, a client device 102 is a smartphone (e.g., iPhone, Android phone, Blackberry, etc.). In certain configurations, a client device 102 can represent an audio player, a game console, a camera, a camcorder, a Global Positioning System (GPS) receiver, a television set top box an audio device, a video device, a multimedia device, and/or a device capable of supporting a connection to a remote server. In some embodiments, a client device 102 can be mobile. In some embodiments, a client device 102 can be stationary. According to certain embodiments, a client device 102 may be a device having at least a processor and memory, where the total amount of memory of the client device 102 could be less than the total amount of memory in a server 106. In some embodiments, a client device 102 does not have a hard disk. In some embodiments, a client device 102 has a display smaller than a display supported by a server 106. In some aspects, a client device 102 may include one or more client devices.

In some embodiments, a server 106 may represent a computer, a laptop computer, a computing device, a virtual machine (e.g., VMware® Virtual Machine), a desktop session (e.g., Microsoft Terminal Server), a published application (e.g., Microsoft Terminal Server), and/or a suitable device with a processor. In some embodiments, a server 106 can be stationary. In some embodiments, a server 106 can be mobile. In certain configurations, a server 106 may be any device that can represent a client device. In some embodiments, a server 106 may include one or more servers.

In some embodiments, a first device is remote to a second device when the first device is not directly connected to the second device. In some embodiments, a first remote device may be connected to a second device over a communication network such as a Local Area Network (LAN), a Wide Area Network (WAN), and/or other network.

When a client device 102 and a server 106 are remote with respect to each other, a client device 102 may connect to a server 106 over the network 104, for example, via a modem connection, a LAN connection including the Ethernet or a broadband WAN connection including DSL, Cable, T1, T3, Fiber Optics, Wi-Fi, and/or a mobile network connection including GSM, GPRS, 3G, 4G, 4G LTE, WiMax or other network connection. Network 104 can be a LAN network, a WAN network, a wireless network, the Internet, an intranet, and/or other network. The network 104 may include one or more routers for routing data between client devices and/or servers. A remote device (e.g., client device, server) on a network may be addressed by a corresponding network address, such as, but not limited to, an Internet protocol (IP) address, an Internet name, a Windows Internet name service (WINS) name, a domain name, and/or other system name. These illustrate some examples as to how one device may be remote to another device, but the subject technology is not limited to these examples.

According to certain embodiments of the subject technology, the terms “server” and “remote server” are generally used synonymously in relation to a client device, and the word “remote” may indicate that a server is in communication with other device(s), for example, over a network connection(s).

According to certain embodiments of the subject technology, the terms “client device” and “remote client device” are generally used synonymously in relation to a server, and the word “remote” may indicate that a client device is in communication with a server(s), for example, over a network connection(s).

In some embodiments, a “client device” may be sometimes referred to as a client or vice versa. Similarly, a “server” may be sometimes referred to as a server device or server computer or like terms.

In some embodiments, the terms “local” and “remote” are relative terms, and a client device may be referred to as a local client device or a remote client device, depending on whether a client device is described from a client side or from a server side, respectively. Similarly, a server may be referred to as a local server or a remote server, depending on whether a server is described from a server side or from a client side, respectively. Furthermore, an application running on a server may be referred to as a local application, if described from a server side, and may be referred to as a remote application, if described from a client side.

In some embodiments, devices placed on a client side (e.g., devices connected directly to a client device(s) or to one another using wires or wirelessly) may be referred to as local devices with respect to a client device and remote devices with respect to a server. Similarly, devices placed on a server side (e.g., devices connected directly to a server(s) or to one another using wires or wirelessly) may be referred to as local devices with respect to a server and remote devices with respect to a client device.

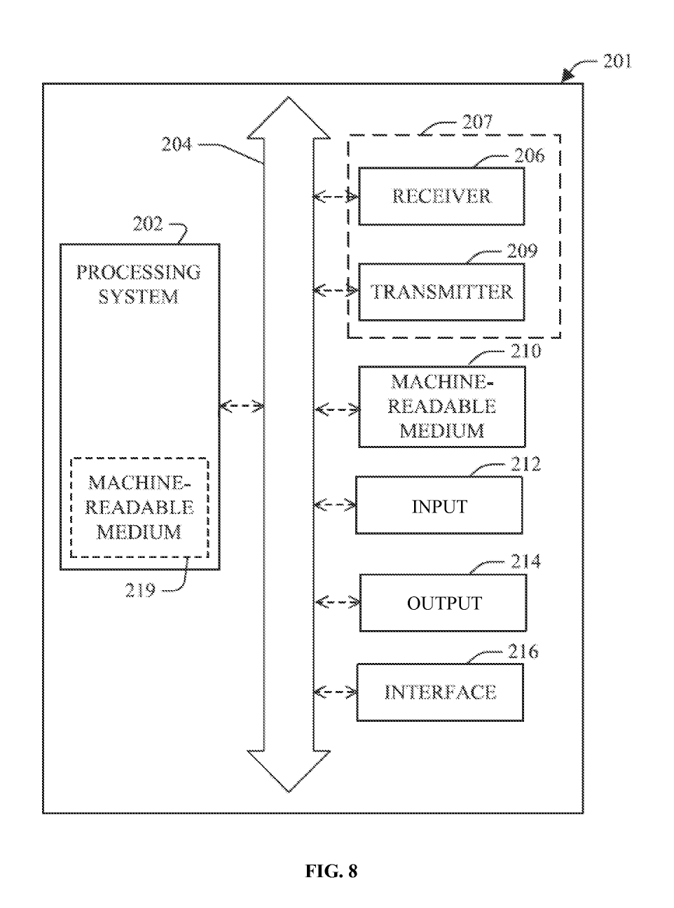

FIG. 8 is a block diagram illustrating an exemplary computer system 201 with which a client device 102 and/or a server 106 of FIG. 7 can be implemented. In certain embodiments, the computer system 201 may be implemented using hardware or a combination of software and hardware, either in a dedicated server, or integrated into another entity, or distributed across multiple entities.

The computer system 201 (e.g., representing one or more of client 102 and/or servers 106 of FIG. 7) includes a bus 204 or other communication mechanism for communicating information, and a processor 202 coupled with the bus 204 for processing information. By way of example, the computer system 201 may be implemented with one or more processors 202. The processor 202 may be a general purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, and/or any other suitable entity that can perform calculations or other manipulations of information.

The computer system 201 can include, in addition to hardware, code that creates an execution environment for the computer program in question, e.g., code that constitutes processor firmware, a protocol stack, a database management system, an operating system, or a combination of one or more of them stored in an included machine-readable medium 210 (e.g. a memory), such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, and/or any other suitable storage device, coupled to the bus 204 for storing information and instructions to be executed by the processor 202. The processor 202 and the memory 204 can be supplemented by, or incorporated in, special purpose logic circuitry.

The instructions may be stored in the machine-readable medium 210 and implemented in one or more computer program products, i.e., one or more modules of computer program instructions encoded on a computer readable medium for execution by, or to control the operation of, the computer system 201, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and/or application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype based languages, off-side rule languages, procedural languages, reflective languages, rule-based languages, scripting languages, stack-based languages, synchronous languages, syntax handling languages, visual languages, wirth languages, and/or xml-based languages. The machine-readable medium 210 may also be used for storing temporary variable or other intermediate information during execution of instructions to be executed by the processor 202; in some embodiments the processor 202 may include a machine-readable medium 219.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

The machine-readable medium 210 of the computer system 201 may further include a data storage device such as a magnetic disk or optical disk, coupled to the bus 204 for storing information and instructions. The computer system 201 may be interfaced to users or to other devices via input 212 and output 214 devices or via interface 216, each of which is connected to bus 204. In addition, computer system 201 may communicate with other computer systems or other types of devices via transceiver 207 (which includes transmitter 209 and receiver 206) using various wired or wireless communication mechanisms. Exemplary input 212, output 214, and interface 216 modules may include data ports (e.g., USB ports), audio ports, and/or video ports, or short-range communication devices such as infrared- or radio-based (e.g. BlueTooth) devices. In some embodiments, the functionality of input 212, output 214, and interface 216 devices may overlap with that of transceiver 207 (e.g. a USB port may be used for connecting input and output devices or for network communications using a USB Ethernet adapter).

In some embodiments, the input/output/interface module may include a communications module. Exemplary communications modules include networking interface cards, such as Ethernet cards, modems, and routers. Exemplary input devices 212 may include a keyboard and/or a pointing device (e.g., a mouse or a trackball) by which a user can provide input to the computer system 201. Other kinds of input devices 212 can be used to provide for interaction with a user as well, such as a tactile input device, visual input device, audio input device, and/or brain-computer interface device. For example, feedback provided to the user can be any form of sensory feedback (e.g., visual feedback, auditory feedback, and/or tactile feedback), and input from the user can be received in any form, including acoustic, speech, tactile, and/or brain wave input. Exemplary output devices 214 may include display devices, such as a cathode ray tube (CRT) or liquid crystal display (LCD) monitor, for displaying information to the user.

According to certain embodiments, a client device 102 and/or server 106 can be implemented using the computer system 201 in response to the processor 202 executing one or more sequences of one or more instructions contained in a machine-readable medium 219 (e.g. memory). Such instructions may be read into the memory from another machine-readable medium 210. Execution of the sequences of instructions contained in the machine-readable medium 219 causes the processor 202 to perform the process steps described herein. One or more processors in a multi-processing arrangement may also be employed to execute the sequences of instructions contained in the machine-readable medium 210, 219. In some embodiments, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component (e.g., a data server), or that includes a middleware component (e.g., an application server), or that includes a front end component (e.g., a client computer having a graphical user interface and/or a Web browser through which a user can interact with an implementation of the subject matter described in this specification), or any combination of one or more such back end, middleware, or front end components. The components of the system 201 can be interconnected by any form or medium of digital data communication (e.g., a communication network). Examples of communication networks include a local area network and a wide area network.

The term “machine-readable storage medium” or “computer readable medium” as used herein (e.g. elements 210, 219) refers to any medium or media that participates in providing instructions to the processor 202 for execution. Such a medium may take many forms, including, but not limited to, non-volatile media, volatile media, and transmission media. Non-volatile media include, for example, optical or magnetic disks. Volatile media include dynamic memory. Transmission media include coaxial cables, copper wire, and fiber optics, including the wires that are part of the bus 204. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As used herein, a “processor” can include one or more processors, and a “module” can include one or more modules.

In an aspect of the subject technology, a machine-readable medium is a computer-readable medium encoded or stored with instructions and is a computing element, which defines structural and functional relationships between the instructions and the rest of the system, which permit the instructions' functionality to be realized. Instructions may be executable, for example, by a system or by a processor of the system. Instructions can be, for example, a computer program including code. A machine-readable medium may include one or more media.

As used herein, the word “module” refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be made of connected logic units, such as gates and flip-flops, and/or may be made of programmable units, such as programmable gate arrays or processors. The modules described herein are preferably implemented as software modules, but may be represented in hardware or firmware.

It is contemplated that the modules may be integrated into a fewer number of modules. One module may also be separated into multiple modules. The described modules may be implemented as hardware, software, firmware or any combination thereof. Additionally, the described modules may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multichip microprocessors, digital signal processors, embedded microprocessors, and microcontrollers.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa.

A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such as “an embodiment” may refer to one or more embodiments and vice versa.

A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

The foregoing description is provided to enable a person skilled in the art to practice the various methods, systems and configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

The subject technology described herein includes a method for facilitating diagnosis of an affective disorder in a person, including the steps of acquiring ¹H spectroscopic data from a region of interest in the brain of the person; analyzing the data by a processor, to obtain an indicator of total choline (tCho) and creatine (Cr) in a region of the brain; and determining, by a processor and based on the indicator, at least one of (i) a likely diagnosis of bipolar disorder or major depressive disorder, or (ii) a probability of bipolar disorder or major depressive disorder, in the person. The method may also include the step of outputting, by a processor, an indicator of the at least one of the likely diagnosis or the probability. The method may also include the step of outputting an indicator of a recommendation that a health care worker consider treating the person in view of the at least one of the likely diagnosis or the probability. In embodiments of the claimed method which include treating the person, the treating may include treating with at least one of a mood stabilizer, lithium, valproic acid, lamotrigine, an atypical antipsychotic agent, an antidepressant, and an SSRI.

In addition, the subject technology described herein includes a method of treating a person, including the steps of treating a person, with at least one medication, based on at least one of (i) a likely diagnosis of bipolar disorder or major depressive disorder, or (ii) a probability of bipolar disorder or major depressive disorder, in the person; acquiring ¹H spectroscopic data from a region of interest in a brain of the person; analyzing the data by a processor, to obtain an indicator of total choline (tCho) and creatine (Cr) in a region of the brain; determining, based on the indicators, at least one of the likely diagnosis or the probability; and outputting, by a processor, an indicator of the at least one of the likely diagnosis or the probability. The medication may be at least one of a mood stabilizer, lithium, valproic acid, lamotrigine, an atypical antipsychotic agent, an antidepressant, and an SSRI.

The methods described herein, directed toward facilitating diagnosis of an affective disorder in a person and treating a person, may be useful wherein the region of interest is the anterior cingulate cortex of the brain. In certain embodiments, the person is an adolescent. Alternatively, the person is an adult. The region of interest may include a voxel, and the ¹H spectroscopic data can be obtained by magnetic resonance spectroscopy.

In some embodiments of the disclosed methods, the person is unmedicated for a mood disorder when acquiring ¹H spectroscopic data from the region of interest in the brain of the person. Alternatively, the person is medicated for a mood disorder when acquiring ¹H spectroscopic data from the region of interest in the brain of the person.

The methods may further include the step of comparing, by a processor, the indicator of total choline (tCho) and creatine (Cr) with an indicator from a data set selected from at least one of a BD data set, a MDD data set, or a normal data set. In certain embodiments, the data set is a normal data set. In addition, the indicator of total choline (tCho) and creatine (Cr) includes at least one of a signal intensity or a concentration of each of total choline (tCho) and creatine (Cr). The disclosed methods may include the further limitation that the analyzing includes calculating a ratio of concentrations and/or signal intensities of the total choline (tCho) and the creatine (Cr); and the determining is based on the ratio of concentrations. The concentration of total choline (tCho) in the disclosed methods may be calculated as a percentage of a total choline signal acquired from the region of interest and the concentration of creatine (Cr) is calculated as a percentage of a creatine signal acquired from the region of interest. Furthermore, the concentration may be an index concentration, and the determining includes comparing the index concentration to a threshold concentration obtained from ¹H spectroscopic data acquired from persons having bipolar disorder, persons having major depressive disorder, and/or persons having neither bipolar disorder nor major depressive disorder.

The subject technology also includes a computer-implemented system for identifying bipolar disorder in a person, the system including: a processing module that processes ¹H spectroscopic data, from a region of interest in a brain of the person, so as to obtain an indicator of total choline (tCho) and creatine (Cr) in a region of the brain; and an output module, in communication with the processing module, that outputs, based on the indicators, a machine-readable indicator of at least one of at least one of (i) a likely diagnosis of bipolar disorder or major depressive disorder, or (ii) a probability of bipolar disorder or major depressive disorder, in the person. The system may have a processing module which is further configured to determine a ratio of concentrations of choline (tCho) and creatine (Cr). In addition, the system may have an output module which is further configured to output, based on the ratio of concentrations, another machine-readable indicator of at least one of the likely diagnosis or the probability. The person in the system may be an adolescent, or may be an adult. The person may also be unmedicated for a mood disorder or may be medicated for a mood disorder. A similar system is disclosed, for a computer-implemented system for identifying major depressive disorder in a person.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

Various features and advantages of the invention are set forth in the following claims.

1. A computer-implemented system for identifying bipolar disorder or major depressive disorder in a person, the system comprising:
a processing module that processes ¹H spectroscopic data, from a region of interest in a brain of the person, so as to obtain an indicator of total choline (tCho) and creatine (Cr) in a region of the brain; and
an output module, in communication with the processing module, that outputs, based on the indicators, a machine-readable indicator of at least one of at least one of (i) a likely diagnosis of bipolar disorder or major depressive disorder, or (ii) a probability of bipolar disorder or major depressive disorder, in the person.

2. (canceled)

3. (canceled)

4. (canceled)

5. (canceled)

6. The system of claim 1, wherein the processing module is further configured to determine a ratio of concentrations of choline (tCho) and creatine (Cr).

7. The system of claim 6, wherein if the ratio is about 0.22, then the person is diagnosed as having bipolar disorder.

8. The system of claim 6, wherein if the ratio is about 0.25, then the person is diagnosed as having major depressive disorder.

9. The system of claim 6, wherein the output module is further configured to output, based on the ratio of concentrations, another machine-readable indicator of at least one of the likely diagnosis or the probability.

10. A method for facilitating diagnosis of an affective disorder in a person, comprising:
acquiring ¹H spectroscopic data from a region of interest in the brain of the person;
analyzing the data by a processor, to obtain an indicator of total choline (tCho) and creatine (Cr) in a region of the brain; and
determining, by a processor and based on the indicator, at least one of
(i) a likely diagnosis of bipolar disorder or major depressive disorder, or
(ii) a probability of bipolar disorder or major depressive disorder, in the person.

11. The method of claim 10, further comprising outputting, by a processor, an indicator of the at least one of the likely diagnosis or the probability.

12. The method of claim 10, further comprising outputting an indicator of a recommendation that a health care worker consider treating the person in view of the at least one of the likely diagnosis or the probability.

13. (canceled)

14. A method of treating a person, comprising:
treating a person, with at least one medication, based on at least one of
(i) a likely diagnosis of bipolar disorder or major depressive disorder, or
(ii) a probability of bipolar disorder or major depressive disorder, in the person;
wherein the likely diagnosis of bipolar disorder or major depressive disorder, or the probability of bipolar disorder to major depressive disorder is determined according to the method of claim 10.

15. (canceled)

16. The method of claim 10, wherein the region of interest is the anterior cingulate cortex of the brain.

17. (canceled)

18. The method of claim 10, wherein the region comprises a voxel.

19. (canceled)

20. (canceled)

21. (canceled)

22. The method of claim 10, further comprising comparing, by a processor, the indicator of total choline (tCho) and creatine (Cr) with an indicator from a data set selected from at least one of a bipolar disorder data set, a major depressive disorder data set, or a normal data set.

23. The method of claim 22, wherein the data set is a normal data set.

24. The method of claim 10, wherein the indicator of total choline (tCho) and creatine (Cr) comprises at least one of a signal intensity or a concentration of each of total choline (tCho) and creatine (Cr).

25. The method of claim 24, wherein the concentration of total choline (tCho) is calculated as a percentage of a total choline signal acquired from the region of interest and the concentration of creatine (Cr) is calculated as a percentage of a creatine signal acquired from the region of interest.

26. The method of claim 24, wherein the concentration is an index concentration, and the determining comprises comparing the index concentration to a threshold concentration obtained from ¹H spectroscopic data acquired from persons having bipolar disorder, persons having major depressive disorder, and/or persons having neither bipolar disorder nor major depressive disorder.

27. The method of claim 24, wherein the analyzing comprises calculating a ratio of concentrations and/or signal intensities of the total choline (tCho) and the creatine (Cr); and the determining is based on the ratio of concentrations.

28. The method of claim 27, wherein if the ratio is about 0.22, then the person is diagnosed as having bipolar disorder.

29. The method of claim 27, wherein if the ratio is about 0.25, then the person is diagnosed as having major depressive disorder.
